(19) United States
(12) Patent Application Publication
(10) Pub. No.: US 2024/0203570 A1
(43) Pub. Date: Jun. 20, 2024

(54) SYSTEMS AND METHODS FOR PRIORITIZING CLINICAL OPPORTUNITIES

(71) Applicant: Change Healthcare Holdings, LLC, Nashville, TN (US)

(72) Inventors: Robert James Ayshford, Chestnut Hill, MA (US); David Michael Jolly, Arlington, TX (US); Tara Lynnmarie Long, Poolville, TX (US); Ananda Nicole Shephard, Haslet, TX (US); Nicholas Eugene Voss, Weatherford, TX (US); Rishi Yagnik, Irving, TX (US)

(21) Appl. No.: 18/068,093

(22) Filed: Dec. 19, 2022

(51) Int. Cl. G16H 40/20

(52) U.S. Cl. CPC G16H 40/20

(57) ABSTRACT

A method for managing clinical opportunities in a pharmacy setting includes aggregating clinical opportunities, which are patient care tasks to be performed by a pharmacy user, from a plurality of sources, ranking the clinical opportunities using one or more ranking parameters, integrating the ranked clinical opportunities into a workflow of one or more pharmacy management systems, determining that a first clinical opportunity from the ranked clinical opportunities has been initiated by a first pharmacy management system of the one or more pharmacy management systems, and updating a current status of the first clinical opportunity within the clinical opportunities repository once the first clinical opportunity is initiated.

### BACKGROUND

More and more often pharmacists are being asked to provide vaccination services and engage in the clinical care of patients, and the role of the pharmacist as patient care provider continues to expand rapidly with expanding legislation. The COVID-19 pandemic, for example, has showcased the value of the pharmacist and pharmacy team, with retail pharmacists playing a pivotal role in the COVID-19 vaccination efforts. However, with increasing demand for additional services (e.g., vaccine administration), pharmacists and technicians are put at risk of burning out. There is new urgency to find workflow efficiencies to reduce stress levels and support expanding clinical and provider status efforts. Additionally, pharmacies continue to see the erosion of medication reimbursement and profit margins from payers and pharmacy benefit managers. There has been movement from financial models based on volume to models based on quality outcomes and keeping patients healthy. Payers are realizing that, when pharmacists are involved with patients, they can have a significant impact on lowering the total cost of care.

### SUMMARY

One implementation of the present disclosure is a patient opportunities management system that includes a processor and memory having instructions stored thereon that, when executed by the processor, cause the system to aggregate clinical opportunities, which are patient care tasks to be performed by a pharmacy user, from a plurality of sources, rank the clinical opportunities using one or more ranking parameters, integrate the ranked clinical opportunities into a workflow of one or more pharmacy management systems, determine that a first clinical opportunity from the ranked clinical opportunities has been initiated by a first pharmacy management system of the one or more pharmacy management systems, and update a current status of the first clinical opportunity within the clinical opportunities repository once the first clinical opportunity is initiated.

Another implementation of the present disclosure is a method that includes aggregating clinical opportunities from a plurality of remote sources, the clinical opportunities being patient care tasks to be performed by a pharmacy user, ranking the clinical opportunities using one or more ranking parameters, integrating the ranked clinical opportunities into a workflow of a plurality of remote pharmacy management systems, determining that a first clinical opportunity from the ranked clinical opportunities has been initiated by a first remote pharmacy management system of the plurality of remote pharmacy management systems, and updating a current status of the first clinical opportunity within the clinical opportunities repository once the first clinical opportunity is initiated.

Yet another implementation of the present disclosure is a non-transitory computer readable medium having instructions stored thereon that, when executed by one or more processors, cause a device to aggregate clinical opportunities, which are patient care tasks to be performed by a pharmacy user, from a plurality of remote sources, rank the clinical opportunities using one or more ranking parameters, integrate the ranked clinical opportunities into a workflow of a plurality of remote pharmacy management systems, determine that a first clinical opportunity from the ranked clinical opportunities has been initiated by a first remote pharmacy management system of the plurality of remote pharmacy management systems, and update a current status of the first clinical opportunity within the clinical opportunities repository once the first clinical opportunity is initiated.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a diagram of an example pharmacy workflow, according to some implementations.

FIG. 2 is a detailed block diagram of a patient opportunities management (POM) system, according to some implementations.

FIG. 3 is a block diagram of a clinical opportunities prioritization communication architecture system, according to some implementations.

FIG. 4 is a flow diagram of a process for prioritizing clinical opportunities, according to some implementations.

FIG. 5 is a block diagram of a communication architecture for updating the status of a clinical opportunity, according to some implementations.

FIG. 6 is a flow diagram of a process for updating the status of a clinical opportunity, according to some implementations.

FIG. 7 is a diagram of an architecture for ranking clinical opportunities, according to some implementations.

FIG. 8 is a flow diagram of a process for filtering or sorting clinical opportunities, according to some implementations.

FIGS. 9-13 are example user interfaces for viewing, searching, and editing ranked clinical opportunities, according to some implementations.

### DETAILED DESCRIPTION

Referring generally to the figures, a system and methods for prioritizing clinical opportunities are shown, accordingly to various implementations. As described herein, a “clinical opportunity” is a patient care task performed by a pharmacist or pharmacy technician outside of their normal role of filling prescriptions. Clinical opportunities can include, for example, medication counseling (e.g., reviewing medications, addressing issues, answering questions, etc.), providing recommendations for patients (e.g., age-based recommendations for medications or supplements), administering immunizations, providing disease management support and education (e.g., medication therapy management), and the like. It will certainly be appreciated that, as available clinical opportunities and patient demand increase, managing available clinical opportunities becomes even more important. Current solutions for managing clinical opportunities, if available, are generally offered outside of a pharmacy's dispensing or management system. For example, currently solutions may provide clinical portals outside of a typical pharmacy workflow, requiring pharmacists to access separate applications for clinical services. This increases the number of steps required of pharmacy users and decreases effectiveness of the intervention, as the pharmacy user needs to make several attempts to reach the patient. In addition, pharmacy users may miss the entire opportunity to interact with the patient if the intervention goes unseen in a separate portal.

In contrast, the system and methods described herein integrate clinical opportunity management with existing pharmacy workflows. For example, the system described herein can be embedded within existing pharmacy technology to provide seamless clinical opportunity integration. Generally, the system and methods described herein provide the ability to rank (e.g., prioritize) clinical opportunities. In some implementations, clinical opportunities are aggregated from a plurality of disparate remote sources (e.g., vendors) and then ranked according to customizable ranking parameters. Ranked clinical opportunities can then be distributed to cooperating pharmacies for fulfillment. Notably, the system and methods described herein allow for accurate, real-time status tracking of available clinical opportunities to coordinate the allocation and completion of opportunities. In this manner, the system and method may improve clinical workflows, pharmacy user experiences, and medication therapy management and other clinical opportunities completion rate efficiencies. In addition, patient outcome and quality measurements may improve, resulting in greater revenue opportunities for pharmacies. Additional features are described in greater detail below.

### Overview

In the most basic pharmacy workflow, prescriptions are filled by a pharmacist or pharmacy technician (collectively referred to herein as “pharmacy users”) and dispensed to patients. Prescriptions are often received from medical providers (e.g., doctors) via fax, phone call, or even as handwritten notes. More recently, pharmacies have implemented pharmacy management systems—also referred to as dispensing systems—to receive electronic prescriptions, maintain customer (e.g., patient) files, and manage the filling/dispensing of pharmaceuticals. Typically, a pharmacy management system receives prescriptions as electronic files (e.g., via electronic prescribing for controlled substances (EPCS)) and manages a queue of pending or in process prescriptions. For example, the pharmacy management system may generate an ordered list of prescriptions to be filled and dispensed based on a variety of criteria, such as time since receipt, availability, urgency, etc. Pharmacy users can then select prescriptions to fill, and, in some cases, the pharmacy management system can track the status of various prescriptions (e.g., being filled, ready for pick up, etc.).

As mentioned above, clinical vendors have begun offering clinical opportunities (e.g., vaccines, medication counseling, etc.) to pharmacy users outside of this typical pharmacy workflow. In some cases, clinical vendors may interact directly with pharmacy users to provide clinical opportunities. For example, clinical vendors may provide pharmacy users with access to a website or remote system in order to view, select, and/or complete clinical opportunities. Thus, it is readily apparent that as clinical service requirements increase for pharmacy users, so too will the number of vendors and/or systems with which pharmacy users need to interact. For example, a pharmacy user that is expected to complete multiple different clinical opportunities each day will likely need to switch between multiple user interfaces and/or systems, greatly reducing efficiency and placing a greater burden on the pharmacy user.

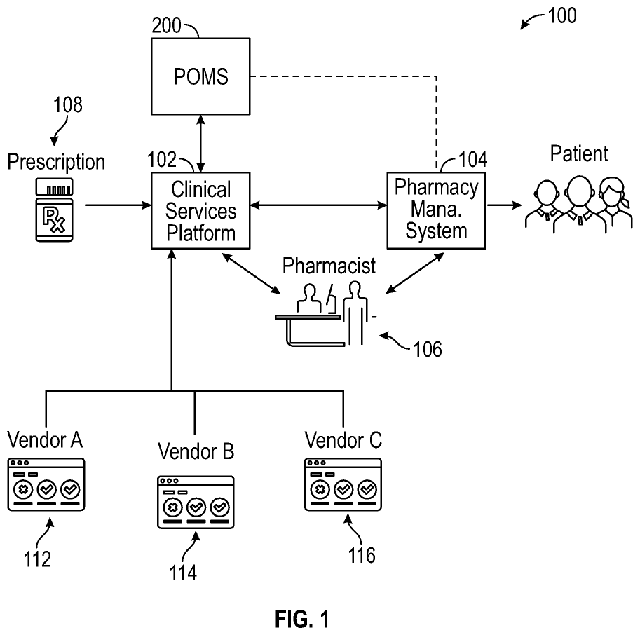

Referring now to FIG. 1, a diagram of an example pharmacy workflow 100 is shown, according to some implementations. Central to workflow 100 are a clinical services platform 102 and a patient opportunities management system (POMS) 200, which may address various shortcomings mentioned above with respect to current systems and/or methods of managing clinical opportunities. As described herein, clinical services platform 102 is generally a system for performing, recording, and billing non-dispensing patient care events, such as comprehensive medical reviews, medication adherence compliance changes, immunizations, and the like. In some implementations, clinical services platform 102 may receive or intercept prescriptions 108, which can be used to inform various decisions and/or controls implemented by clinical services platform 102. For example, clinical services platform 102 may use prescription data to determine whether a comprehensive medical review should be conducted with a patient or may determine when to schedule follow-up appointments or phone calls. In some implementations, clinical services platform 102 communicates with (e.g., sends data to and/or receives data from) a pharmacy management system 104. For example, clinical services platform 102 may transmit notifications (e.g., alerts), forward prescriptions, send control signals or commands, etc., to pharmacy management system 104 and may receive responses or other data from pharmacy management system 104.

Pharmacy management system 104—alternatively a pharmacy dispensing system or enterprise pharmacy system—is generally a system for managing dispensing tasks within a pharmacy. For example, pharmacy management system 104 may maintain or access patient pharmacy records to store or retrieve prescriptions, retrieve patient information (e.g., provider identifiers, insurance information, etc.), and the like. In some implementations, pharmacy management system 104 generates and displays a list of pharmacy tasks (e.g., prescriptions to be filled) that can be viewed and/or selected by a pharmacy user. In some implementations, pharmacy users can use pharmacy management system 104 to access patient information for filling prescriptions, handling pickups, billing for prescriptions, etc. It should be appreciated that, in some implementations, pharmacy management system 104 may directly receive prescriptions 108 from healthcare providers. In some such implementations, pharmacy management system 104 may communicate received prescriptions 108 to clinical services platform 102 and/or clinical services platform 102 may access pharmacy management system 104 to retrieve prescription information. In some implementations, as mentioned above, clinical services platform 102 may intercept prescriptions 108 as they are transmitted to pharmacy management system 104. In other implementations, clinical services platform 102 may not have access to prescription information.

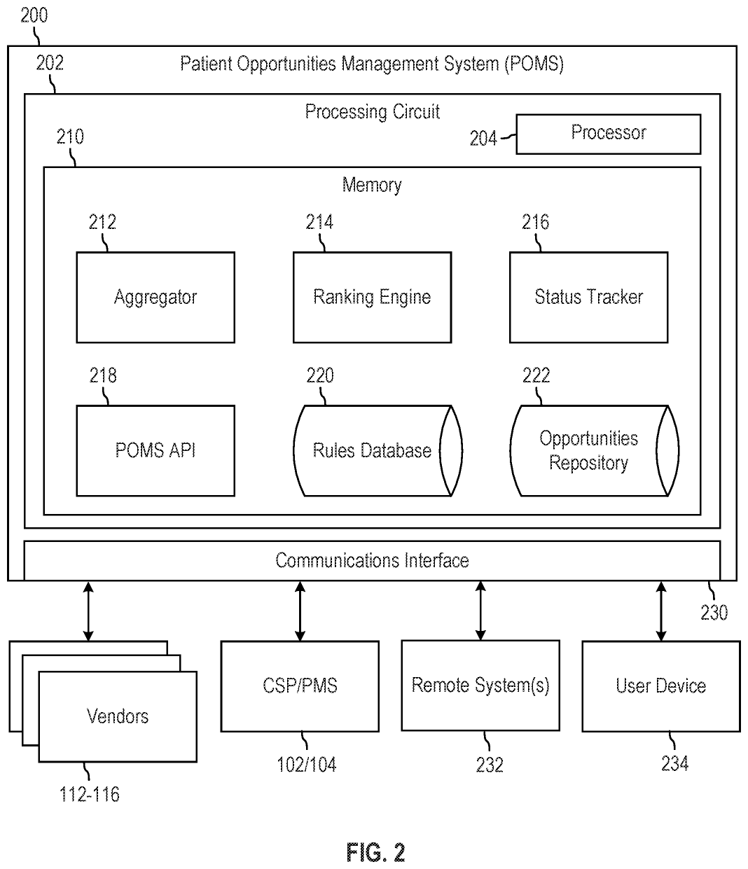

POMS 200, as described in greater detail below with respect to FIG. 2, is generally a system for managing clinical opportunities in a pharmacy setting. In particular, POMS 200 is generally configured to aggregate and rank clinical opportunities from a plurality of sources. Clinical opportunities can generally be ranked by any number of criteria, which may be selected by a user. For example, clinical opportunities may be ranked by average reimbursement value, average time to complete, age of the opportunity, the number of additional opportunities that a given clinical opportunity could lead to, pharmacy user role, etc. In the example shown, the sources of clinical opportunities are vendors 112-116. While only three vendors are shown, it should be appreciated that POMS 200 may receive or retrieve and aggregate clinical opportunities from any number of sources.

In some implementations, such as the one shown in FIG. 1, vendors 112-116 may transmit clinical opportunities first to clinical services platform 102, from which the clinical opportunities are forwarded to POMS 200. In other implementations, as mentioned below, clinical services platform 102 and POMS 200 are implemented on the same computing device and/or POMS 200 is part of clinical services platform 102; thus, POMS 200 may directly access or receive clinical opportunities via clinical services platform 102. In yet other implementations, POMS 200 may receive clinical opportunities directly from vendors 112-116. Similarly, in some implementations, pharmacy management system 104 may communicate directly with POMS 200, rather than communicating with POMS 200 exclusively though clinical services platform 102.

In some implementations, POMS 200 works cooperatively with clinical services platform 102 to manage non-billing pharmacy tasks and integrate clinical opportunities into a pharmacy's usual workflow. In other implementations, POMS 200 can work independently of clinical services platform 102 to integrate clinical opportunities into a pharmacy's workflow. In some implementations, POMS 200 manages the distribution of clinical opportunities to multiple pharmacies (e.g., multiple of pharmacy management system 104). For example, POMS 200 may maintain a repository of clinical opportunities and may manage how the clinical opportunities are assigned to various pharmacy management systems to prevent two or more pharmacy management systems from attempting to retrieve and complete the same clinical opportunity. Notably, POMS 200 integrates clinical opportunities into a pharmacy's workflow via the pharmacy's management system (e.g., pharmacy management system 104) by “injecting” clinical opportunities between other pharmacy tasks (e.g., prescriptions). In this manner, pharmacy users are provided with a single source of both dispensing and non-dispensing tasks (e.g., clinical opportunities) within a single interface.

As shown, a pharmacy user 106 (e.g., a pharmacist) may interact with one or both of clinical services platform 102 and pharmacy management system 104. For example, in some implementations, pharmacy user 106 may interact with clinical services platform 102 for non-dispensing tasks and pharmacy management system 104 for dispensing tasks. Alternatively, while note shown, pharmacy user 106 may interact with POMS 200 directly. In some implementations, clinical services platform 102, pharmacy management system 104, and/or POMS 200 can be accessed via a common user interface or device. For example, in some such implementations, clinical services platform 102 and pharmacy management system 104 may both be accessed via a pharmacy's workstation or computer, which is operable by pharmacy user 106. In some implementations, pharmacy user 106 only directly interacts with clinical services platform 102 to change settings or parameters, define rules, etc., but otherwise interacts mainly with pharmacy management system 104. Put another way, pharmacy user 106 may generally interact with clinical services platform 102 through pharmacy management system 104 but may also have the ability to interact directly with clinical services platform 102 as needed. For example, pharmacy user 106 may primarily interact with pharmacy management system 104 to view both dispensing and non-dispensing tasks (e.g., dispensing prescriptions to patients, performing medication reviews, etc.). As shown, clinical services platform 102 may interact with pharmacy management system 104 to provide an integrated experience to pharmacy user 106.

In various implementations, each of clinical services platform 102, pharmacy management system 104, and POMS 200 may be, or may be hosted on, distinct computing devices. For example, clinical services platform 102 and POMS 200 may each by hosted on a separate server. As another example, pharmacy management system 104 may be, or may be hosted on, a pharmacy's workstation or computer. It should be appreciated that clinical services platform 102, pharmacy management system 104, and POMS 200 may be implemented by any suitable computing device. In general, each computing device may have at least one processor and memory, as described in greater detail below with respect to FIG. 2. It should also be appreciated that, while clinical services platform 102, pharmacy management system 104, and POMS 200 are shown as singular components in workflow 100, any number of clinical services platforms, pharmacy management system, and/or patient opportunities management systems may be interconnected to operate as described herein. For example, in some implementations, a single clinical services platform 102 and POMS 200 may manage non-dispensing tasks for a plurality of pharmacy management systems.

While not shown, in some implementations, clinical services platform 102, pharmacy management system 104, and/or POMS 200 may be communicably coupled by any suitable network or direct connection. For example, clinical services platform 102 and pharmacy management system 104 may communicate via a wired or wireless network, such as a virtual private network (VPN), local area network (LAN), wide area network (WAN), etc. In some implementations, pharmacy management system 104 communicates with clinical services platform 102 via the Internet. In some implementations, clinical services platform 102 and POMS 200 are each part of the same system or are each hosted on the same computing device. For example, clinical services platform 102 and POMS 200 may be hosted on a common server. In some implementations, POMS 200 is a component of clinical services platform 102. Thus, it should be appreciated that the specific layout or positioning of components in workflow 100 is not intended to be limiting.

### Patient Opportunities Management System

Referring now to FIG. 2, a detailed block diagram of POMS 200 is shown, according to some implementations. As mentioned above, POMS 200 is generally configured to aggregate and rank clinical opportunities, which can then be integrated into a pharmacy workflow via the pharmacy's management system (e.g., pharmacy management system 104). More specifically, POMS 200 obtains clinical opportunities from a plurality of vendors 112-116, ranks each of the clinical opportunities according to one or more ranking parameters, and distributes the ranked clinical opportunities to one or more pharmacy management systems, where the clinical opportunities are integrated into the pharmacy's normal workflow. In some implementations, POMS 200 is further configured to assign clinical opportunities to patients, either automatically or based on user inputs. For example, POMS 200 may automatically determine which patients to assign incoming opportunities and/or may receive inputs from a user of POMS 200 that assigns opportunities to patients.

For clarity, POMS 200 is described herein as a separate component from clinical services platform 102. For example, POMS 200 may be a system or program that can be accessed by clinical services platform 102 but that is not part of clinical services platform 102. However, as also mentioned above, it should be appreciated that POMS 200 can be implemented as a component of clinical services platform 102 or may be implemented via the same computing device as clinical services platform 102. For example, the functionality of POMS 200 described herein may be implemented by clinical services platform 102 such that, to an end user, POMS 200 is not distinct from clinical services platform 102.

POMS 200 is shown to include a processing circuit 202 that includes a processor 204 and a memory 210. Processor 204 can be a general-purpose processor, an application-specific integrated circuit (ASIC), one or more field programmable gate arrays (FPGAs), a group of processing components, or other suitable electronic processing structures. In some embodiments, processor 204 is configured to execute program code stored on memory 210 to cause POMS 200 to perform one or more operations, as described below in greater detail. It will be appreciated that, in embodiments where POMS 200 is part of another computing device (e.g., clinical services platform 102), the components of POMS 200 may be shared with, or the same as, the host device. For example, if POMS 200 is implemented via clinical services platform 102, then POMS 200 may utilize the processing circuit, processor(s), and/or memory of clinical services platform 102 to perform the functions described herein.

Memory 210 can include one or more devices (e.g., memory units, memory devices, storage devices, etc.) for storing data and/or computer code for completing and/or facilitating the various processes described in the present disclosure. In some embodiments, memory 210 includes tangible (e.g., non-transitory), computer-readable media that stores code or instructions executable by processor 204. Tangible, computer-readable media refers to any physical media that is capable of providing data that causes POMS 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include RAM, ROM, hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some embodiments, POMS 200 may be implemented within a single computing device (e.g., one server, one housing, etc.). In other embodiments, POMS 200 may be distributed across multiple servers or computers (e.g., that can exist in distributed locations). For example, POMS 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. For example, virtualization software may be employed by POMS 200 to provide the functionality of a number of servers that is not directly bound to the number of computers in POMS 200.

Memory 210 is shown to include an aggregator 212, which is generally configured to aggregate clinical opportunities obtained (e.g., received and/or retrieved) from a plurality of vendors 112-116. As described above, vendors 112-116 can include any number of clinical vendors that generate and/or provide clinical opportunities. For example, vendors 112-116 may be pharmaceutical companies, government or regulatory agencies, healthcare providers, etc. In some implementations, clinical opportunities are communicated from vendors 112-116 to POMS 200, and thereby aggregator 212, via a communications interface 230, described in greater detail below. In some such implementations, clinical opportunities are obtained via a POM application programming interface (API) 218, which acts as an interface between POMS 200 and any external devices or systems (e.g., vendors 112-116). In some implementations, clinical opportunities are communicated as data packets that contain information about each clinical opportunity, such as the opportunity type, monetary value, timing, etc. In some implementations, clinical opportunities are received as links that can be used by aggregator 212 to navigate to a remote system (e.g., one of vendors 112-116) to retrieve clinical opportunity data.

In some cases, clinical opportunities are obtained from vendors 112-116 in various different formats and/or may contain disparate information. Accordingly, in some such implementations, aggregator 212 is further configured to convert obtained clinical opportunities into a common format. For example, as part of the aggregation process, aggregator 212 may convert a file or data type of each clinical opportunity to a common format for subsequent analysis and modifications. In some implementations, aggregator 212 can identify a relevant subset of information contained within each clinical opportunity for subsequent ranking and/or presentation to a user. In some implementations, clinical opportunities are stored in an opportunities repository 222 (e.g., a database or other storage location). Opportunities repository 222 is generally configured to maintain a plurality of clinical opportunities. It should be appreciated that the clinical opportunities stored in opportunities repository 222—or, more broadly, obtained by POMS 200—are not necessarily intended for any specific pharmacy. Rather, opportunities repository 222 can maintain clinical opportunities that can be accessed or executed by any number of different pharmacies.

In some implementations, clinical opportunities are obtained from vendors 112-116 as generic “programs” that include various information and parameters associated with the clinical opportunity, but that are not assigned to individual patients. For example, a medication review for a particular drug may be provided by the drug's manufacturer, but medication review may not be assigned to particular patients. Instead, in some such implementations, aggregator 212 may be further configured to assign clinical opportunities to individual patients. Alternatively, aggregator 212 may simply store the clinical opportunity programs in opportunities repository 222. In some implementations, a user may then define various priority settings or clinical opportunity parameters to the programs and/or certain settings may be automatically defined. Parameters can include, for example, a program name, originating vendor, primary user role (e.g., for completing the clinical opportunity), average time to complete, average reimbursement, whether the clinical opportunity leads to additional clinical opportunities, and whether the clinical opportunity leads to or requires additional prescriptions. In some implementations, parameters set as the program name and vendor can be automatically defined once the clinical opportunity is received, but parameters such as the average time to complete, average reimbursement, etc., may be user-defined to ensure that clinical opportunities are vendor agnostic.

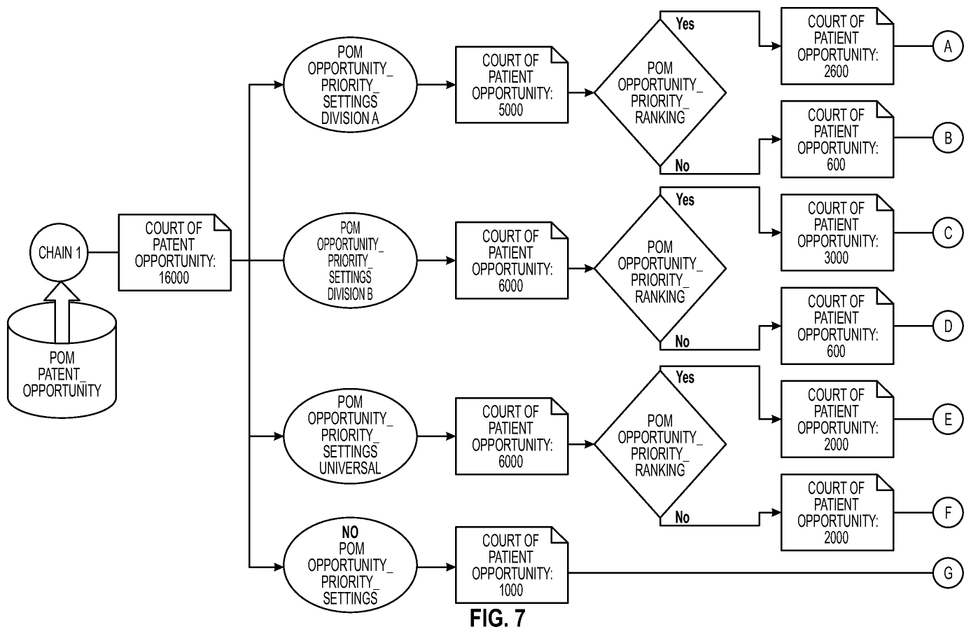

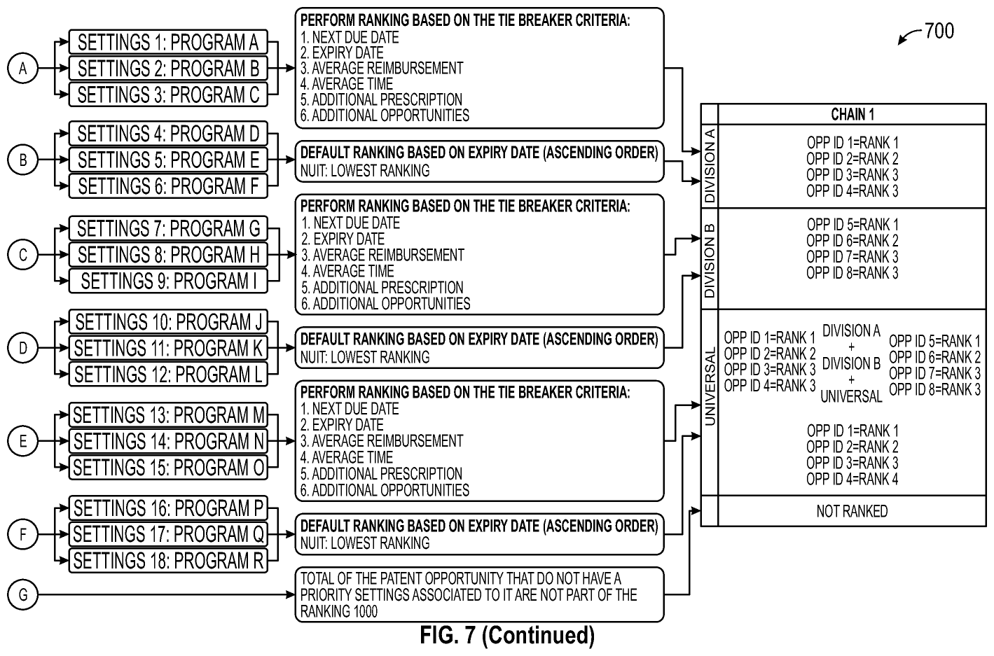

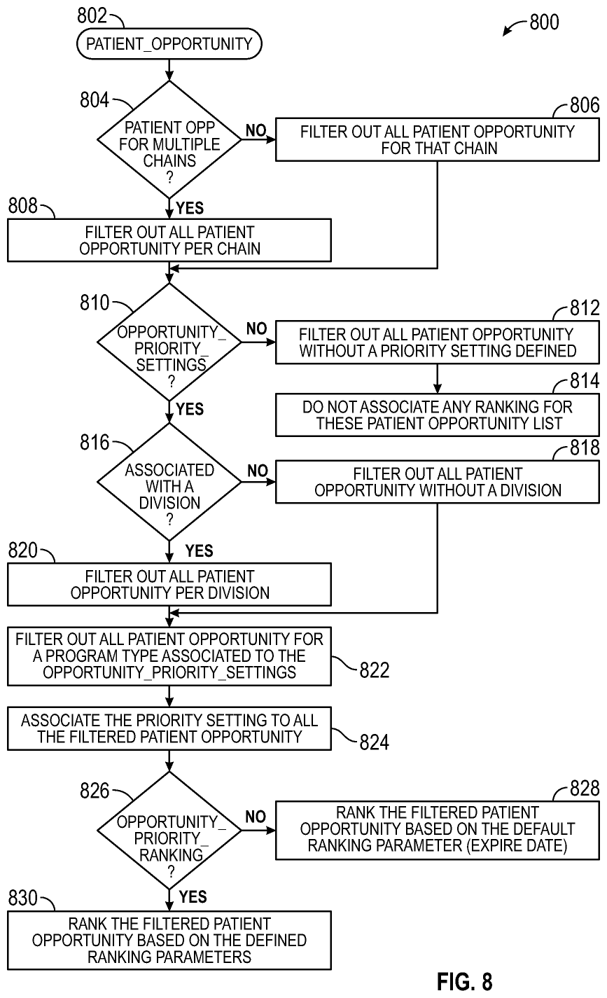

After the clinical opportunities are aggregated and/or assigned to patients, a ranking engine 214 is configured to rank the aggregate clinical opportunities based on various ranking parameters stored in a rules database 220. As described herein, ranking parameters can be any user-defined parameters that can be used to order (i.e., rank) clinical opportunities. Ranking parameters can include, for example, average reimbursement value, average time to complete, whether the clinical opportunity will result in additional clinical opportunities, whether the clinical opportunity will result in additional prescriptions, due dates or deadlines for completion, expiration date, and the like. In some implementations, various ranking parameters can be established as priority settings. For example, average reimbursement value may be a higher priority than average time to completion. The ranking of clinical opportunities is described in greater detail below with respect to FIGS. 7 and 8.

As mentioned above, POMS 200 is generally configured to aggregate, rank, and otherwise maintain clinical opportunities for any number of clients (e.g., pharmacies). In other words, POMS 200 can interface with, or is accessible to, a number of different client devices, such as pharmacy management systems (e.g., pharmacy management system 104), and therefore acts as a “centralized” clinical opportunities manager. Accordingly, in some implementations, memory 210 includes a status tracker 216 that tracks and updates the status of each clinical opportunity in opportunities repository 222. For example, status tracker 216 can determine when clinical opportunities are sent to and/or initiated by a remote pharmacy and can update a status of the clinical opportunity within opportunities repository 222 (e.g., to “in progress” or “assigned”). In other words, status tracker 216 manages the distribution of clinical opportunities. This way, status tracker 216 can ensure two or more pharmacies do not attempt to complete the same clinical opportunity.

Another key feature of POMS 200 is the integration of clinical opportunities into a pharmacy's normal workflow. In other words, POMS 200 can facilitate the integration of clinical opportunities into a pharmacy's task list, such that pharmacy users do not need to navigate between different systems, user interfaces, etc., to find, view, and complete clinical opportunities. In some implementations, clinical opportunities are “injected” or inserted between other pharmacy tasks in a pharmacy's task list, such as dispensing tasks. In some such implementations, POMS 200 itself is configured to perform the integration of clinical opportunities. For example, POMS 200 may intercept prescription and/or otherwise determine the “other” pharmacy tasks for each pharmacy and may insert clinical opportunities between these other pharmacy tasks. In some implementations, each pharmacy's management or computing system performs the actual integration of clinical opportunities. In some such implementations, each pharmacy's management or computing system may continuously or periodically access or communicate with POMS 200 to retrieve their respective clinical opportunities. For example, in some implementations, external systems and devices may interact with POMS 200 via POMS API 218 in order to receive a continuous or periodic feed of clinical opportunities data.

Generally, POMS API 218 facilitates communications between POMS 200 and any of vendors 112-116, clinical services platform 102, pharmacy management system 104, and user device 234. For example, POMS API 218 can allow external and/or remote systems (e.g., pharmacy management system 104) to access opportunities repository 222 to retrieve clinical opportunities. In some implementations, as mentioned above, POMS API 218 facilitates the continuous or periodic transfer of clinical opportunities data to each of one or more client devices (e.g., pharmacy management systems). In some implementations, POMS API 218 is a REST API, but it should be appreciated that POMS API 218 may be any suitable type of API.

POMS 200 is also shown to include a communications interface 230 that facilitates communications between POMS 200 and any external components or devices, including clinical services platform 102, pharmacy management system 104, vendors 112-116, remote systems 232, and/or user device 234. For example, communications interface 230 can provide means for transmitting data to, or receiving data from, pharmacy management system 104. Accordingly, communications interface 230 can be or can include a wired or wireless communications interface (e.g., jacks, antennas, transmitters, receivers, transceivers, wire terminals, etc.) for conducting data communications, or a combination of wired and wireless communication interfaces. In some embodiments, communications via communications interface 230 are direct (e.g., local wired or wireless communications) or via a network (e.g., a WAN, the Internet, a cellular network, etc.). For example, communications interface 230 may include one or more Ethernet ports for communicably coupling POMS 200 to a network (e.g., the Internet). In another example, communications interface 230 can include a WiFi© transceiver for communicating via a wireless communications network. In yet another example, communications interface 230 may include cellular or mobile phone communications transceivers.

In some implementations, POMS 200 is communicably coupled to user device 234 via communications interface 230 (e.g., via a wireless network). User device 234 may be a computing device including a memory (e.g., RAM, ROM, Flash memory, hard disk storage, etc.), a processor (e.g., a general-purpose processor, an ASIC, one or more FPGAs, a group of processing components, or other suitable electronic processing components), and a user interface (e.g., a touch screen), allowing a user to interact with POMS 200. User device 234 can include, for example, one or more mobile phones, electronic tablets, laptops, desktop computers, workstations, and other types of electronic devices. More generally, user device 234 may include any electronic device that allows a user to interact with POMS 200, and more broadly with POMS 200, by presenting and/or receiving user inputs through a user interface. In some implementations, user device 234 represents a user interface positioned on POMS 200 itself. In some implementations, user device 234 is configured to execute (i.e., run) a software application that presents the various user interfaces described herein.

Remote system(s) 232, as described herein, may include any systems that are not included in the aforementioned list, but that may also interact with POMS 200 (e.g., via POMS API 218). For example, remote system(s) 232 may include third-party pharmacy management system (e.g., other than pharmacy management system 104), user devices, or the like. In some implementations, remote system(s) 232 may include websites, databases, or other devices and/or systems that POMS 200 may access to collect information about clinical opportunities, to report completed opportunities, etc.

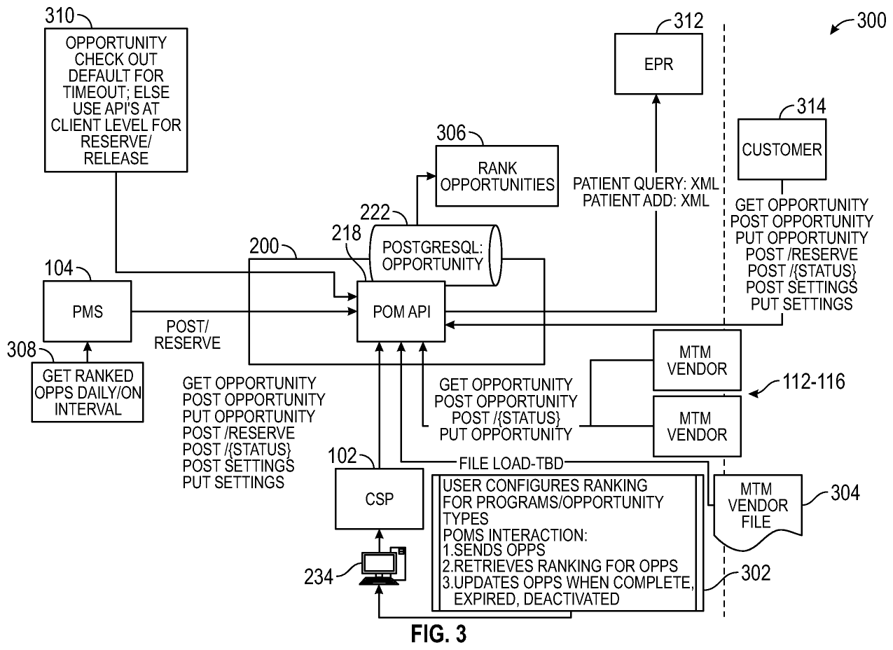

Referring now to FIG. 3, a block diagram of a clinical opportunities prioritization communication architecture 300 is shown, according to some implementations. At a high level, FIG. 3 shows the various components of workflow 100, discussed above, and the various information/data communicated from/to each component. In other words, communication architecture 300 is a detailed representation of workflow 100, showing an example process for obtaining and distributing clinical opportunities. At the center of FIG. 3 is POMS 200 which, as described above, is generally configured to aggregate, rank, and manage clinical opportunities. In this example, each external component (e.g., external to POMS 200) communicates with POMS 200 via POMS API 218. In some implementations, POMS API 218 uses a REST API architecture. Accordingly, in some such implementations, communication architecture 300 illustrates the various HTTP methods used by each component of workflow 100. However, it should be appreciated that POMS API 218 is not necessarily a REST API but may use any suitable API architecture or language. For example, POMS API 218 may by implemented via GraphQL, Falcor, etc.

Looking initially to the bottom of FIG. 3, configuration parameters 302 may be uploaded, entered, or otherwise provided to clinical services platform 102 via user device 234. Accordingly, configuration parameters 302 are user defined. In some implementations, user device 234 is a workstation or other type of computing device operated by a user of POMS 200. In some implementations, user device 234 is a workstation or other type of computing device at a pharmacy. Configuration parameters 302 generally include parameters for ranking and/or prioritizing clinical opportunities. As mentioned above, ranking parameters can include, for example, average reimbursement value, average time to complete, whether the clinical opportunity will result in additional clinical opportunities, whether the clinical opportunity will result in additional prescriptions, due dates or deadlines for completion, expiration date, and the like. In some implementations, a user may select various predefined parameters for ranking and/or prioritizing clinical opportunities. In some implementations, a user may define custom parameters for ranking and/or prioritizing clinical opportunities. In some implementations, a user may define a “weight” or value associated with each parameter.

As mentioned above, POMS 200 generally obtains clinical opportunities from a plurality of vendors 112-116. In this example, vendors 112-116 may transmit and/or receive clinical opportunities to/from POMS 200. For example, a GET call is used to retrieve resources from POMS 200 (e.g., via POMS API 218). These resources may be clinical opportunities, information about clinical opportunities, information about the pharmacies or patients that use POMS 200, etc. As another example, a POST call can be used to post new clinical opportunities or statuses for various opportunities. In some implementations, vendor files 304 may be loaded onto POMS 200 via POMS API 218. For example, vendors 112-116 may transmit vendor files 304 and/or POMS 200 may retrieve vendor files 304. Received and/or retrieved vendor files 304, which generally contain information about one or more clinical opportunities, may be stored in opportunities database 222, as discussed above. POMS 200 is generally configured to rank the clinical opportunities contained therein based on the user-defined ranking parameters (e.g., part of configuration parameters 302) and may store ranked clinical opportunities 306 in opportunities database 222.

In some implementations, pharmacy management system 104 communicates with POMS 200 to retrieve 308 ranked clinical opportunities 306. For example, pharmacy management system 104 may retrieve ranked clinical opportunities 306 at a regular interval (e.g., daily), continuously (e.g., as new clinical opportunities become available), or on-demand (e.g., based on a user prompt). In some such implementations, pharmacy management system 104 may send an API call (e.g., a request) to POMS API 218 which causes POMS 200 to transmit a subset of available clinical opportunities. In some implementations, pharmacy management system 104 may directly access opportunities repository 222 (e.g., a repository) to retrieve ranked clinical opportunities 306. As noted above, it should be appreciated that pharmacy management system 104 can generally include any number of pharmacy management systems; thus, the description of only a single pharmacy management system herein is not intended to be limiting.

In some implementations, the status of retrieved clinical opportunities is updated within opportunities repository 222 upon being retrieved by, or otherwise transmitted to, pharmacy management system 104. For example, pharmacy management system 104 may make a POST call to POMS API 218 to set the status of retrieved clinical opportunities to “reserved”, thereby preventing other devices or systems from obtaining the same clinical opportunities. Put another way, client level devices (e.g., pharmacy management system 104) may reserve/release clinical opportunities within opportunities repository 222 via API calls or the like. In some implementations, a timeout threshold may be set for each clinical opportunity that is “checked out” or released to a client device (e.g., pharmacy management system 104). In this manner, clinical opportunities may be automatically “checked in” or released by the corresponding client device if not completed or initiated within the designated time. For example, the timeout threshold may be set to 24 hours, such that any clinical opportunities that are reserved by pharmacy management system 104 for more than 24 hours are automatically released back to POMS 200 to be made available to other users.

In some implementations, POMS 200 can retrieve and/or transmit patient information from/to an electronic patient records (EPR) database 312. For example, POMS 200 may retrieve patient information in order to identify relevant clinical opportunities (e.g., a medication review based on the patient's prescriptions). As another example, POMS 200 may update a patient record based on completed clinical opportunities (e.g., receiving a vaccine). In some implementations, customers 314 may be able to directly interact with POMS 200 in a similar manner to vendors 112-116 and/or pharmacy management system 104. Customers 314 may include, for example, pharmacy users (e.g., pharmacist), pharmacies, insurance companies, etc. As shown, customers 314 may be able to reserve clinical opportunities for completion. For example, a customer that does not utilize a pharmacy management system may still be able to access POMS 200 in order to obtain and complete clinical opportunities.

To this point, it should be appreciated that POMS 200 is generally able to be operated as a stand-alone system (e.g., separate from a pharmacy management system 104 or clinical services platform 102). For example, POMS 200 may aggregate, rank, and maintain clinical opportunities separately from any of the other components of workflow 100 described above. In some implementations, POMS 200 is remotely accessible such that a user can view, initiate, and/or perform clinical opportunities from any computing device. For example, a user may access POMS 200 via a computer that is separate from a pharmacy management system. Notably, POMS 200 may also be integrated with any clinical platform or pharmacy management system, and not just those described herein. For example, any third-party system (e.g., pharmacy management systems, clinical platforms, etc.) may access POMS 200 via POMS API 218.

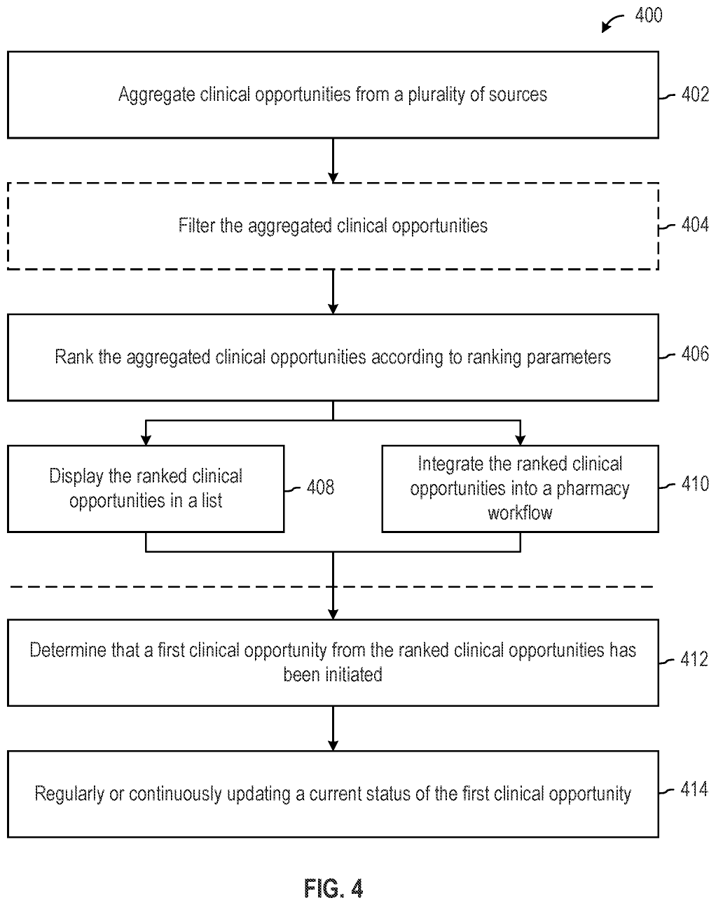

Referring now to FIG. 4, a flow diagram of a process 400 for ranking clinical opportunities is shown, according to some implementations. In some implementations, process 400 is implemented by POMS 200, as described above. Alternatively, in some implementations, process 400 is partially or fully implemented by clinical services platform 102. It will be appreciated that certain steps of process 400 may be optional and, in some implementations, process 400 may be implemented using less than all of the steps shown. It will also be appreciated that the order of steps shown in FIG. 4 is not intended to be limiting.

At step 402, clinical opportunities are aggregated from a plurality of sources. As described above, clinical opportunities are patient care tasks performed by a pharmacy user (e.g., a pharmacist or pharmacy technician) outside of his/her roll in dispensing prescriptions. Clinical opportunities can include, for example, medication counseling (e.g., reviewing medications, addressing issues, answering questions, etc.), providing recommendations for patients (e.g., age-based recommendations for medications or supplements), administering immunizations, providing disease management support and education (e.g., medication therapy management), and the like. In general, clinical opportunities are generated and/or provided by various vendors (i.e., “sources”), such as pharmaceutical manufacturers, regulator or compliance bodies, healthcare providers, insurance companies, etc. Generally, these vendors are remote from POMS 200 and/or clinical services platform 102 but may have an agreement or contract in place with the entity that operates POMS 200 and/or clinical services platform 102 in order to provide clinical opportunities.

As described herein, “aggregating” clinical opportunities generally includes obtaining one or more clinical opportunities from one or more sources and storing the obtained clinical opportunities in a repository or database (e.g., opportunities repository 222). In some implementations, clinical opportunities are received or retrieved from various sources (e.g., vendors 112-116). For example, clinical opportunities may be periodically (e.g., daily) transmitted from various vendors to POMS 200. As another example, POMS 200 may periodically request clinical opportunities from various vendors. In any case, the repository or database may be configured to maintain the aggregate clinical opportunities. In some implementations, clinical opportunities are obtained in disparate formats and/or may contain disparate information. In some such embodiments, POMS 200 may be configured to normalize the clinical opportunities by converting each clinical opportunity to a common type and/or by selecting a subset of information from each clinical opportunity to use in subsequent steps of process 400.

At step 404, the aggregated clinical opportunities are optionally filtered according to one or more filtering criteria. In some implementations, the filtering criteria is predefined or selected by a user. Filtering criteria can include, for example, opportunity type, priority setting, geographical location, pharmacy identifier, patient identifier, etc. As an example, clinical opportunities may be filtered according to a geographical region in which a patient associated with the clinical opportunity lives. In another example, clinical opportunities may be filtered by pharmacy location, division, store number, etc. In yet another example, clinical opportunities may be filtered based on the associated patient. Filtering is described in greater detail below with respect to FIG. 8. It should also be appreciated that clinical opportunities may be filtered at any point in process 400. In some implementations, filtering the clinical opportunities includes sorting or separating the clinical opportunities based on the associated patient, pharmacy, region, etc. In some such implementations, POMS 200 may be configured to identify relevant information in each clinical opportunity for sorting or separating. In some implementations, POMS 200 can add a corresponding identifier or other information to each clinical opportunity.

At step 406, the aggregated clinical opportunities are ranked according to ranking parameters. Ranking, or “prioritizing,” the clinical opportunities generally includes ordering the aggregated clinical opportunities based on various ranking parameters or criteria. In some implementations, ranking parameters are user-defined and/or may be selected by a user from a predefined list. Ranking parameters can include, for example, average reimbursement value, average time to complete, whether the clinical opportunity will result in additional clinical opportunities, whether the clinical opportunity will result in additional prescriptions, due dates or deadlines for completion, expiration date, and the like. Accordingly, relevant information may be extracted from each clinical opportunity and compared to the ranking parameters (e.g., stored in a database) to assign a value, rank, position, or weight to each clinical opportunity. In some implementations, multiple ranking parameters are used, in which case each ranking parameter may have a different value that affects how the clinical opportunities are ranked.

Assume, for example, that average reimbursement value, average time to complete, and expiration date are the three ranking parameters being used to evaluate a first set of clinical opportunities. Assume also that a user has selected average reimbursement value as the most important parameter and average time to complete as the least important parameter. Accordingly, average reimbursement value may be given additional weight or may have a greater value when ranking multiple clinical opportunities. In this example, a first clinical opportunity has an average reimbursement value of $50, an average completion time of 30 minutes, and an expiration date 90 days from the current date, and a second clinical opportunity has an average reimbursement value of $25, an average completion time of 10 minutes, and an expiration date 30 days from the current date. In some cases, the first clinical opportunity may be ranked higher than (e.g., prioritized over) the second clinical opportunity, since the average reimbursement value is much higher. Alternatively, the combined value of the lower average completion time and shorter expiration period of the second clinical opportunity may cause the second clinical opportunity to be ranked higher than the first clinical opportunity.

Because ranking criteria are generally user-defined, ranking criteria may vary for each user/pharmacy/etc. For example, a first pharmacy may define a first set of ranking criteria, which may be different from a second set of ranking criteria defined by a second pharmacy. Accordingly, clinical opportunities may first be filtered into groups (e.g., at step 404) before ranking. In some such implementations, each group of clinical opportunities is ranked separately. For example, a first subset of the aggregated clinical opportunities associated with a particular pharmacy or store may be ranked separately from a second subset of the aggregated clinical opportunities associated with a different pharmacy or store.

Once ranked, the clinical opportunities may be maintained in a repository (e.g., opportunities repository 222) until they are transmitted to, or retrieved by, one or more client devices. A “client device” is any computing device that is capable of accessing or interacting with POMS 200 in order to obtain clinical opportunity information. One such client device is a pharmacy management system (e.g., pharmacy management system 104) or another computing device at a pharmacy. In a retail setting, the client device may be a server or other computing device at a store which has a pharmacy. In some implementations, the ranked clinical opportunities are subsequently communicated collectively (e.g., in a batch) or individually to various client devices. In some implementations, clinical opportunities are only accessible by associated client devices or pharmacies (e.g., based on the filtering parameters discussed above). For example, clinical opportunities for a first patient may only be available to pharmacies that are within a certain distance of the first patient's home address, usual pharmacy, or last-visited pharmacy. In other implementations, any client device is capable of obtaining the clinical opportunities.

In some implementations, process 400 continues to step 408 after the clinical opportunities are ranked. In other implementations, process 400 continues to step 410, described below. In yet other implementations, both steps 408 and 410 may be implemented. At step 408, the ranked clinical opportunities are displayed in a list. More generally, the ranked clinical opportunities may be collectively maintained in opportunities repository 222 such that any client device can access the aggregated and ranked clinical opportunities. For example, a user (e.g., a pharmacy user using a pharmacy computer) may access a complete or partial list of available clinical opportunities from a web page or other user interface. In some implementations, only a subset of available clinical opportunities are available to a user based on the filtering parameters discussed above. In some implementations, a list of clinical opportunities is transmitted to each client device. In some such implementations, the list of clinical opportunities may be a subset of all the ranked clinical opportunities associated with that client device, pharmacy, user, etc.

Alternatively, or additionally, at step 410, the ranked clinical opportunities are integrated into a pharmacy's workflow. Accordingly, ranked clinical opportunities may be transmitted to, or retrieved by, a plurality of client devices (e.g., pharmacy computers) after being ranked. In some implementations, opportunities repository 222, or at least a portion of opportunities repository 222, is accessible by client devices. For example, one or more client devices (e.g., pharmacy management system 104) may remotely access opportunities repository 222 to “retrieve” clinical opportunities or may request clinical opportunities from POMS 200 (e.g., via an API call). In some implementations, clinical opportunities are automatically transmitted to their respective destinations (e.g., pharmacy management system or other client devices) after being ranked. In some implementations, POMS API 218 serves as a gateway for client devices to periodically or continuously receive clinical opportunities. For example, client devices (e.g., pharmacy management system 104) may interface with POMS 200 via POMS API 218 to receive periodic or continuous updates. In some such embodiments, client devices may “subscribe” to clinical opportunity updates via POMS 200, such that new clinical opportunities are populated on the client device as they are obtained, filtered, and/or ranked by POMS 200.

As described herein, integrating clinical opportunities into a pharmacy's workflow generally includes injecting clinical opportunities between other pharmacy tasks (e.g., dispensing tasks) within a pharmacy's management system. In this way, clinical opportunities will be seamlessly presented within a pharmacy's task list. For example, clinical opportunities can be displayed between other pharmacy tasks such that the clinical opportunities can be selected and/or completed between other pharmacy tasks. In some implementations, clinical opportunities can be automatically initiated, or queued for initiation, based on their positioning within the pharmacy management task list. For example, a clinical opportunity may be a “next task” for a pharmacy user after completing a currently active task (e.g., dispensing a medication). In some implementations, initiating a clinical opportunity causes a client device (e.g., a pharmacy workstation) to automatically navigate to and/or display a user interface (e.g., website, application, etc.) associated with the vendor that provided the clinical opportunity. For example, if a pharmaceutical manufacturer provided a clinical opportunity for a medication review of a particular prescription, then initiating the clinical opportunity may automatically navigate the user to a medication review webpage on the pharmaceutical manufacturer's website.

Generally, clinical opportunities are presented within a pharmacy's task list based on their ranking (e.g., assigned at step 406). For example, higher priority opportunities may be placed near the top of the pharmacy's task list, while lower priority opportunities are placed nearer the bottom. In some implementations, clinical opportunities are injected into a pharmacy's task list as openings within the task list are detected. For example, as dispensing and other pharmacy tasks are completed, clinical opportunities may be injected based on their ranking. In some implementations, a first subset of the clinical opportunities for each pharmacy are initially integrated into the pharmacy's task list and a second subset of the clinical opportunities are reserved for available openings or “down time.” For example, one or more higher priority opportunities with designated timeframes or scheduled completion times may be integrated into the pharmacy's task list at the beginning of the day, and additional lower priority opportunities are reserved until the pharmacy's initial task list is completed.

At step 412, a determination is made that a first clinical opportunity has been initiated. In some implementations, the determination is based on a communication from the client device (e.g., pharmacy management system 104) that initiated the first clinical opportunity. For example, responsive to a user selecting the first clinical opportunity from a pharmacy's task list, the pharmacy's management system may send a notification to POMS 200 indicating that the first clinical opportunity has been initiated. In some implementations, the determination is based on a status of the first clinical opportunity within opportunities repository 222. For example, the status of the first clinical opportunity may be set to “reserved” or “checked out” when the first clinical opportunity is sent to, or retrieved by, a pharmacy's management system.

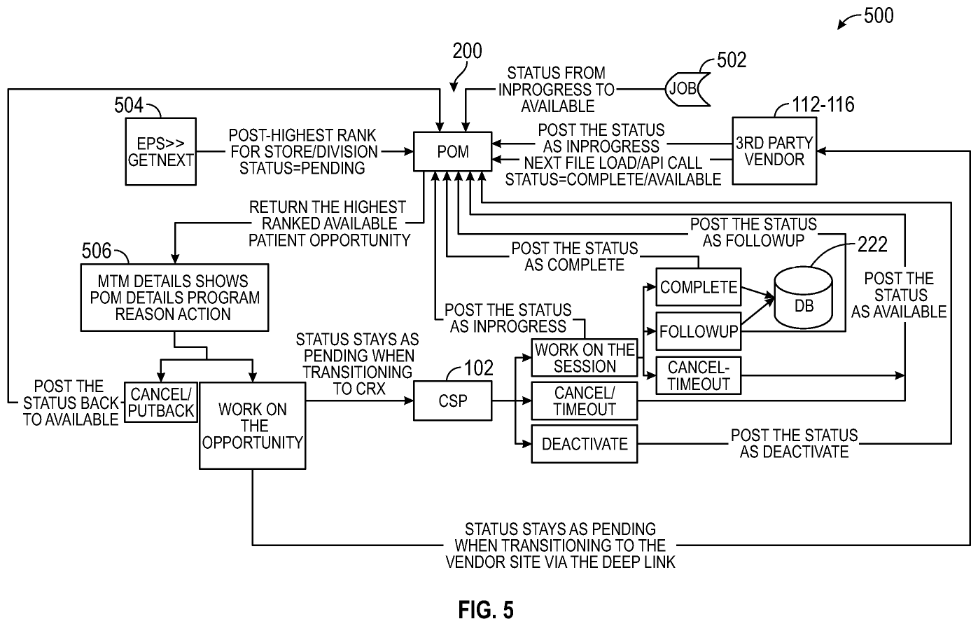

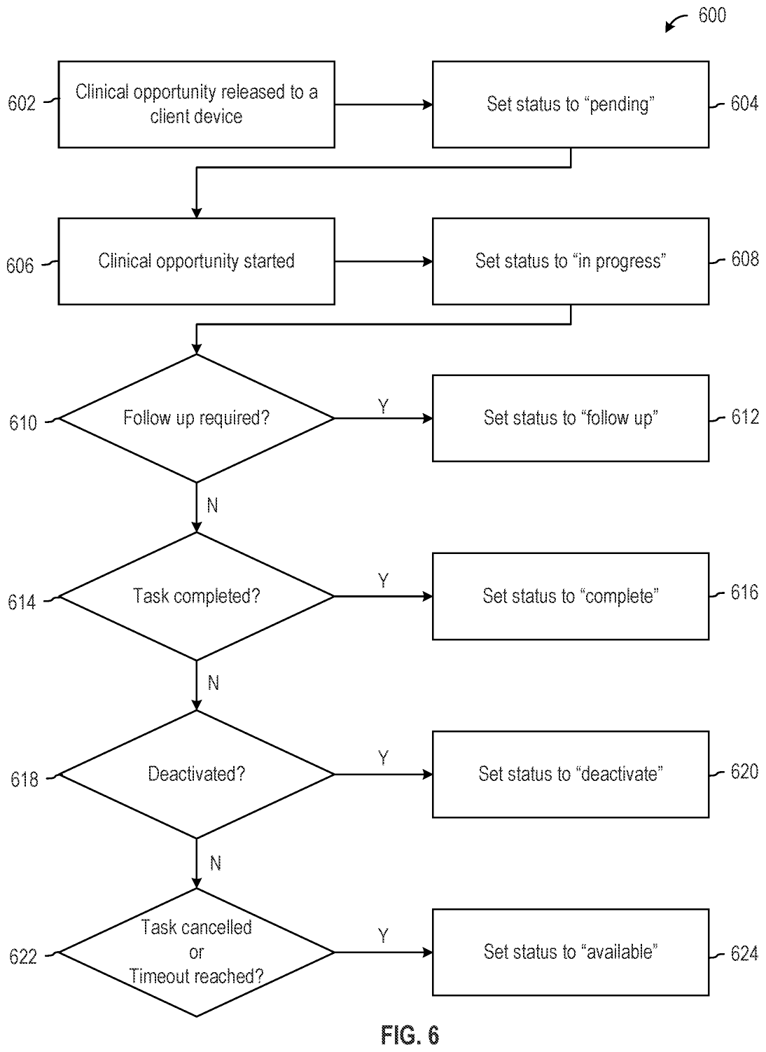

At step 414, a current status of the first clinical opportunity is regularly or continuously updated. In some implementations, the status of the first clinical opportunity is updated once it is determined that the first clinical opportunity has been initiated. For example, the status of the first clinical opportunity may be changed from “not active” to “initiated”. In some implementations, the status of the first clinical opportunity is updated as a user (e.g., a pharmacist) completes the first clinical opportunity. For example, the first clinical opportunity's status may simply change to “completed” once the first clinical opportunity is completed, or the first clinical opportunity may include multiple checkpoints or milestones that can be marked completed by a user, in which case the first clinical opportunity's status may provide additional detail regarding the state of the first clinical opportunity. A more detailed explanation of the process of maintaining a status of each clinical opportunity is described in greater detail below with respect to FIGS. 5 and 6.

### Example Use Case

To better understand the aggregation, ranking, and distribution of clinical opportunities via POMS 200, an example use case is described herein. Assume, for this example, that POMS 200 obtains clinical opportunities from three different vendors. Upon receipt, POMS 200 may combine and store these clinical opportunities into opportunities repository 222 (step 402). Optionally, POMS 200 may normalize or convert any of the clinical opportunities that do not fit a required or preferred format. In some cases, POMS 200 may also identify a target pharmacy, store, or patient for each clinical opportunity, and may sort or “filter” the clinical opportunities within opportunities repository 222 or prior to being stored in opportunities repository 222. For example, POMS 200 may separate the clinical opportunities into different lists, packets, or databases within opportunities repository 222.

Assume, for this example, that POMS 200 manages clinical opportunities for three different pharmacies. POMS 200 may separate the aggregate clinical opportunities based on their respective destination pharmacies. In some implementations, POMS 200 may determine a target pharmacy based on information contained within each clinical opportunity. For example, POMS 200 may look for a store identifier or a patient identifier, which can be used to determine which pharmacy or store the clinical opportunity is intended to be sent to. POMS 200 may then rank the clinical opportunities in individual groups. For example, POMS 200 may sort the clinical opportunities into three sets (e.g., one for each destination pharmacy) and may rank each set of clinical opportunities separately, based on the respective ranking parameters for their destination pharmacies. Alternatively, one or more sets of clinical opportunities may be ranked using the same ranking parameters.

After ranking, POMS 200 may simply store the ranked clinical opportunities in opportunities repository 222 for later retrieval by each of the three pharmacies or, more specifically, by the pharmacy management system(s) associated with each of the three pharmacies. In this case, each pharmacy may retrieve their respective ranked clinical opportunities at any point. Alternatively, POMS 200 may transmit each set of ranked clinical opportunities to its respective pharmacy. For example, each of the three destination pharmacies may utilize a pharmacy management system that interfaces with POMS 200 via POMS API 218, such that each destination pharmacy can receive continuous or periodic updates of clinical opportunities. The respective clinical opportunities can then be integrated directly into the respective pharmacy's task list or “workflow.”

### Managing Clinical Opportunities

Referring now to FIG. 5, a block diagram of a communication architecture 500 for updating the status of a clinical opportunity is shown, according to some implementations. As mentioned repeatedly herein, POMS 200 is generally configured to aggregate and maintain clinical opportunities from a plurality of sources (e.g., vendors) and can provide ranked clinical opportunities to a plurality of client devices (e.g., pharmacy management systems). To avoid multiple client devices reserving or “checking out” the same clinical opportunities, POMS 200 may track and update a status of each clinical opportunity (e.g., in opportunities repository 222) as it is distributed, initiated, and completed. In some implementations, this status tracking is performed by status tracker 216, but is generally described herein as being a function of POMS 200.

As shown, POMS 200 may obtain clinical opportunities (shown as “jobs” 502) and may initially set a status of each clinical opportunity to “available”. In some cases, client devices (e.g., pharmacy management system 104) may retrieve one or more clinical opportunities by transmitting, to POMS 200, a request 504 (e.g., a “GetNext” command). In some implementations, a request is transmitted and/or clinical opportunities are retrieved responsive to a pharmacy user initiating a dispensing task. In some implementations, upon receiving a request 504, POMS 200 returns the highest ranked available clinical opportunity 506. In some implementations, the highest ranked available clinical opportunity 506 is the highest ranked opportunity for the respective client device. In other implementations, the highest ranked available clinical opportunity 506 is simply the next highest ranked clinical opportunity. In any case, POMS 200 may transmit, to the client device, details of the clinical opportunity. In addition, POMS 200 may change the status of the clinical opportunity from “available” to “pending.” Upon receipt, the clinical opportunity may either by returned to POMS 200 via a cancel or putback request, or the clinical opportunity may be initiated. If the clinical opportunity is cancelled or “put back,” the status may be updated from “in progress” to “available.”

If instead the clinical opportunity is initiated, the status may remain as “pending” until a subsequent action is taken. Thus, “pending” simply indicates that the clinical opportunity is “checked out” or released to a client device, but that the clinical opportunity has not been started. However, once started, the clinical opportunity's status may be changed to “in progress.” From there, the clinical opportunity may be completed (e.g., a status of “complete”), a follow up may be scheduled (e.g., a status of “follow up”), or the clinical opportunity may be cancelled. In some implementations, the clinical opportunity may also reach a timeout threshold in which case the clinical opportunity may be automatically cancelled (e.g., made available). For example, the timeout threshold could be 24 hours to ensure that clinical opportunities do not sit with a client device for too long without being completed. Alternatively, instead of working on the clinical opportunity, a user of the client device may deactivate the clinical opportunity, which may update the status to “deactivated.” Unlike simply cancelling the clinical opportunity, which sets the status to “available,” deactivating a clinical opportunity may prevent the clinical opportunity from being released to another client device.

In some implementations, vendors 112-116 may receive status updates for each associated clinical opportunity. For example, vendors 112-116 may be notified when a clinical opportunity is set to “pending.” In some implementations, initiating a clinical opportunity may require a user to navigated to a vendor website or application to complete the clinical opportunity, in which case vendors 112-116 may also submit status updates to POMS 200. For example, if a user is actively working on a clinical opportunity within a vendor's website, the vendor may post a status of “in progress.” Alternatively, when the clinical opportunity is completed via the vendor's website or application, the clinical opportunity may be marked as “complete.” In some implementations, vendors 112-116 may automatically load a new clinical opportunity onto POMS 200 based on a determination that a clinical opportunity is complete.

Referring now to FIG. 6, a flow diagram of a process 600 for updating the status of a clinical opportunity is shown, according to some implementations. In some aspects, process 600 follows and builds upon the description of FIG. 5, above. In some implementations, process 600 is implemented by POMS 200, as described above. Alternatively, in some implementations, process 600 is partially or fully implemented by clinical services platform 102. It will be appreciated that certain steps of process 600 may be optional and, in some implementations, process 600 may be implemented using less than all of the steps shown. It will also be appreciated that the order of steps shown in FIG. 6 is not intended to be limiting.

At step 602, a clinical opportunity is released to a client device (e.g., a pharmacy management system). As mentioned above, in some implementations, the clinical opportunity is released responsive to a request from the client device. In other implementations, the clinical opportunity is automatically assigned and released to the clinical device. Upon being released, at step 604, the status of the clinical opportunity is set to “pending.” Subsequently, at step 606, a determination is made that the clinical opportunity has been started or is being worked on. In some implementations, this determination is made based on an indication or notification received from the client device. For example, the client device may transmit a notification once a user selects the clinical opportunity from a list (e.g., a task list). Accordingly, at step 608, the status of the clinical opportunity is set to “in progress.”

Once the clinical opportunity is in progress, a variety of determination can be made to update the clinical opportunity's status. In some implementations, each determination is made based on data (e.g., notifications) received from the client device. At step 610, for example, a determination is made as to whether a follow up is required. If so, the status of the clinical opportunity is set to “follow up” at step 612. If not, then at step 614, a determination is made as to whether the clinical opportunity is complete. If so, the status of the clinical opportunity is set to “complete” at step 616. If not, then at step 618, a determination is made as to whether the clinical opportunity has been deactivated by a user of the client device. If so, the status of the clinical opportunity is set to “deactivated” at step 620. If not, then at step 622, a determination is made as to whether the clinical opportunity has been cancelled or the timeout threshold has been reached. If either is true, the status of the clinical opportunity is set to “available” at step 624.

Referring now to FIG. 7, a diagram of an example architecture 700 for ranking clinical opportunities is shown, according to some implementations. In particular, architecture 700 is an example ranking schema for a chain of pharmacies (“Chain 1”). In this example, 16,000 clinical opportunities associated with the chain of pharmacies are being ranked. As shown, the chain of pharmacies generally includes two divisions—“Division A” and “Division B”—into which the clinical opportunities can be organized. These divisions may be groups of pharmacies that share a characteristic, such as geographical location, name or type, etc. Accordingly, in some implementations, the clinical opportunities may be sorted into one of “Division A” or “Division B”. Alternatively, the clinical opportunities may each have an identifier or other information that designates which division it belongs to. For example, the clinical opportunities may be presorted or defined for a respective division (e.g., based on distance to a patient, etc.). Some clinical opportunities may be considered “universal” and are therefore not associated with a particular division. For example, a vaccine appointment for a first patient may be available to any pharmacy within the chain, thereby allowing the patient to visit any pharmacy to receive their vaccine.

In this example, the original 16,000 clinical opportunities are split between “Division A”, “Division B”, and universal, in groups of 5,000. An additional 1,000 clinical opportunities do not have any priority settings or identifiers, so they will not be ranked. However, the remaining clinical opportunities may be ranked according to various ranking parameters associated with each division. Looking at “Division A,” for example, each clinical opportunity may be analyzed to determine whether it is to be ranked using tie breaker criteria or whether it will be ranked using a default method. In some implementations, clinical opportunities may include a priority identifier or other indicator that identifies the clinical opportunity as “important” or otherwise. In this example, 2,500 opportunities are identified for priority ranking, and are thus ranked according to ranking parameters (e.g., “tie breaker” criteria). For example, these ranking criteria may be next due date, expiry date, average reimbursement, average time, additional opportunities, or additional prescriptions. Any clinical opportunities that are not identified for priority ranking may be ranked according to a default scheme, such as by expiry date.

Referring now to FIG. 8, a flow diagram of a process 800 for filtering (e.g., sorting) and ranking clinical opportunities is shown, according to some implementations. In some aspects, process 800 is better understood with reference to FIG. 7, above. In some implementations, process 800 is implemented by POMS 200, as described above. Alternatively, in some implementations, process 800 is partially or fully implemented by clinical services platform 102. It will be appreciated that certain steps of process 800 may be optional and, in some implementations, process 800 may be implemented using less than all of the steps. It will also be appreciated that the order of steps shown in FIG. 8 is not intended to be limiting.

At step 802, a plurality of client opportunities are obtained. Subsequently, at step 804, a determination is made, for each client opportunity, as to whether the client opportunity is available for multiple chains or “groups” of pharmacies. This determination may be made, for example, based on an identifier associated with each client opportunity or based on other information obtained from an associated vendor of each client opportunity. If a client opportunity is not available for multiple chains, then at step 806, the clinical opportunity may be sorted or “filtered out” into its associated chain. Otherwise, at step 808, any “universal” client opportunities, or client opportunities available to multiple chains, may be filtered out or sorted.

At step 810, a determination is made as to whether each client opportunity has a defined priority setting or identifier. If a client opportunity does not have a defined priority setting or identifier, then at step 812, the client opportunity is filtered out and, at step 814, is added to a separate list of client opportunities that will not be ranked. If, however, the client opportunity does have a defined priority setting or identifier, then at step 816, a second determination is made as to whether each client opportunity is associated with a division (e.g., a group of pharmacies). If a client opportunity is not associated with a division, then it may be filtered out at step 818. Otherwise, at step 820, the client opportunities associated with divisions are separately filtered. Subsequently, at step 822, all the client opportunities are filtered based on program time. At step 824, predefined ranking criteria are then associated with any remaining filtered client opportunities. At step 826, a determination is made as to whether each client opportunity has a priority ranking identifier, which is an indicator or identifier that prioritizes the clinical opportunity or otherwise marks it for ranking based on ranking criteria. If a clinical opportunity does not have said identifier, then at step 828, it may be ranked accordingly to a default parameter (e.g., expiry date). Otherwise, at step 830, the remaining clinical opportunities are ranked based on the predefined ranking parameters.

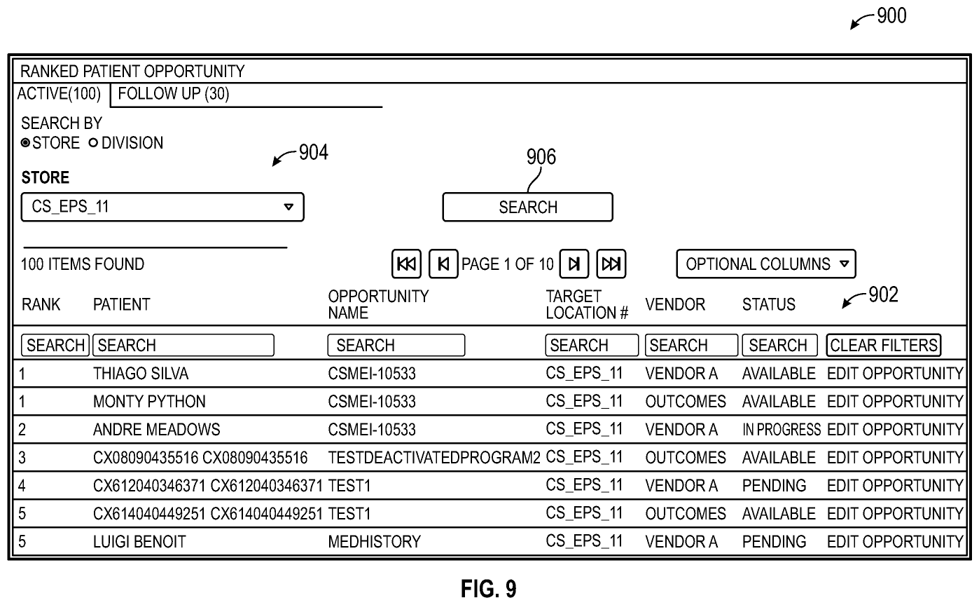

Referring now to FIGS. 9-13, various example user interfaces for viewing, searching, and editing ranked clinical opportunities are shown, according to some implementations. Turning first to FIG. 9, an example user interface 900 is shown for viewing all available ranked clinical opportunities. In particular, interface 900 is shown to include a ranked clinical opportunities list 902. In this example, list 902 contains 100 clinical opportunities that have been ranked by POMS 200. Each entry in list 902 is shown to include a rank, a patient name, an opportunity name, a target location (e.g., a destination pharmacy or client deice), a vendor identifier, and a status. Additionally, list 902 is shown to include a plurality of text entry boxes for searching for specific opportunities. For example, a user could enter a patient name to identify any clinical opportunities that are for that patient. Interface 900 also includes various filtering parameters 904 for filtering the clinical opportunities shown in list 902. For example, the user could filter or search by store, by selecting a “store” icon and selecting a particular store identifier from a drop down menu. Alternatively, the user could search by division. Once a store or division is selected, the user may select a “search” button 906 to filter list 902 to the selected store or division.

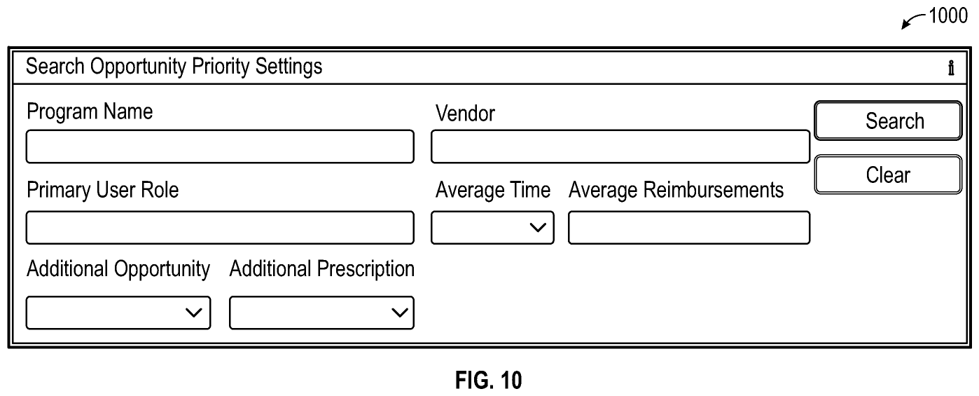

Referring now to FIG. 10, an example user interface 1000 for searching clinical opportunity programs based on their priority settings is shown, according to some implementations. A clinical opportunity “program,” as described herein, is a defines various parameters of a clinical opportunity such that the clinical opportunity can be assigned to multiple different patients. These parameters, also called “priority settings,” are user-defined and include characteristics such as a program name, originating vendor, primary user role (e.g., for completing the clinical opportunity), average time to complete, average reimbursement, whether the clinical opportunity leads to additional clinical opportunities, and whether the clinical opportunity leads to or requires additional prescriptions. Notably, user defined clinical opportunity parameters (e.g., priority settings) allow clinical opportunity rankings to be vendor agnostic, as all clinical opportunities can be ranked based on predefined criteria.

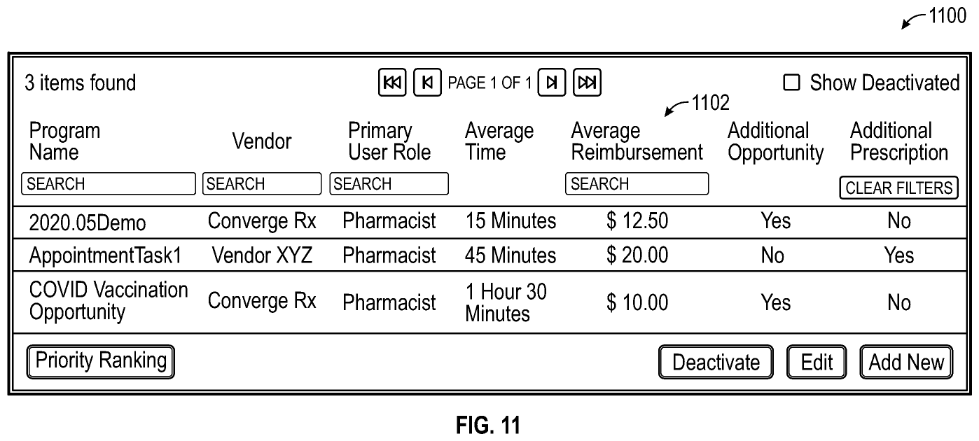

In this example, interface 1000 includes multiple text entry fields in which a user can enter text for searching for clinical opportunities. For example, a user could search by program name, vendor, user role, average time, etc. The results of said search may yield the example user interface 1100 shown in FIG. 11. For example, interface 1100 may be a search for all clinical opportunity programs with a primary user role of “pharmacist.” Alternatively, a user may navigate to interface 1100 by another manner, such as by selecting a link from a menu. In yet another alternative, interface 1000 may be displayed as an overlay to interface 1100.

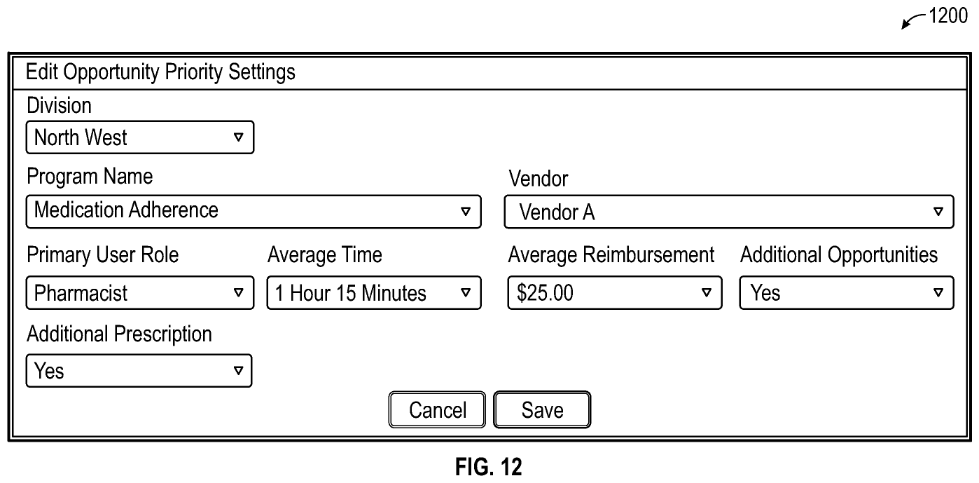

Interface 1100 includes a list 1102 of clinical opportunities and their respective priority settings. For example, list 1102 includes a program name, vendor name, user role for completing the opportunity, average time, average reimbursement, etc. From interface 1100, a user can edit, deactivate, or add new priority settings for a selected clinical opportunity listed. For example, a user may select an “Add New” icon to define new priority settings or may select an “Edit” icon to edit an existing entry. FIG. 12 shows an example user interface 1200 for editing and/or establishing priority settings by selecting one of the icons from interface 1100. As shown, a user may define various clinical opportunity parameters including a relevant division or store, program name, vendor, primary user role, average time, etc. In some implementations, various parameters are predefined and are therefor selected from menus or lists. For example, the clinical opportunity programs and vendors are generally known and cannot be manually entered by a user. Thus, the user may select a specific program from a drop-down menu in order to edit other fields (e.g., average time, average reimbursement, etc.).

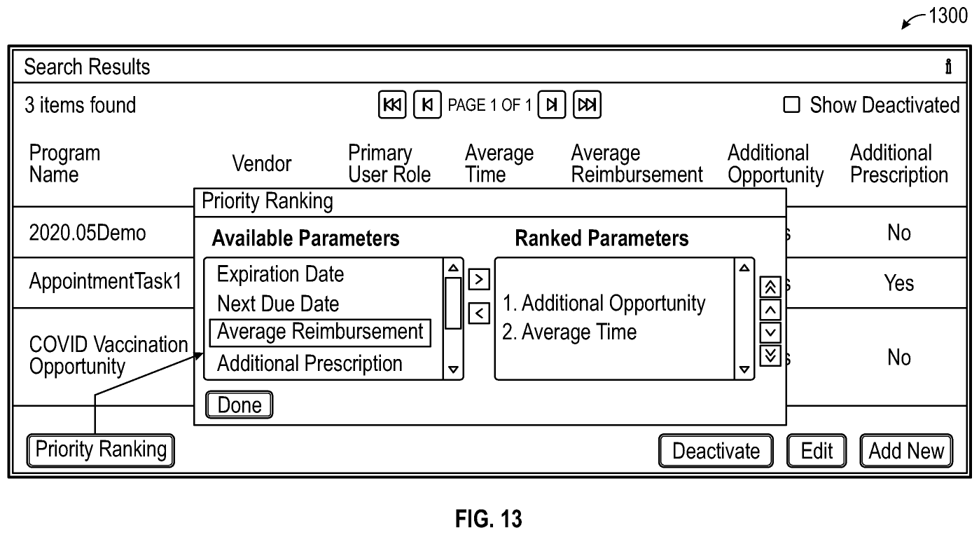

Referring now to FIG. 13, an example user interface 1300 for establishing priority ranking parameters is shown, according to some implementations. As shown, a user may navigate to interface 1300 via interface 1100, such as by selecting a “Priority Ranking” icon. Interface 1300 provides a list of available ranking parameters, such as expiration date, next due date, average reimbursement, etc. A user can then select which ranking parameters to apply to clinical opportunities when performing the ranking described herein. In some implementations, the user may further define an order of the parameters, or a “priority”. In this example, the ranking parameter for “additional opportunities” (e.g., whether completing the clinical opportunity leads to more clinical opportunities) is the most important ranking parameter, and thus would take precedence over any other ranking parameters.

### Configuration of Certain Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A patient opportunities management system comprising:
a processor; and
memory having instructions stored thereon that, when executed by the processor, cause the system to:
aggregate clinical opportunities from a plurality of sources, wherein the clinical opportunities are patient care tasks to be performed by a pharmacy user;
rank the clinical opportunities using one or more ranking parameters;
integrate the ranked clinical opportunities into a workflow of one or more pharmacy management systems;
determine that a first clinical opportunity from the ranked clinical opportunities has been initiated by a first pharmacy management system of the one or more pharmacy management systems; and
update a current status of the first clinical opportunity within the clinical opportunities repository once the first clinical opportunity is initiated.

2. The system of claim 1, wherein integrating the ranked clinical opportunities into the workflow of the plurality of remote pharmacy management systems comprises:
identifying a subset of the ranked clinical opportunities associated with each of the plurality of remote pharmacy management systems; and
distributing each subset of the ranked clinical opportunities to a respective one of the plurality of remote pharmacy management systems to be integrated with respective workflows of the one of the plurality of remote pharmacy management systems.

3. The system of claim 1, wherein integrating the ranked clinical opportunities into a workflow of one or more pharmacy management systems comprises injecting the ranked clinical opportunities between other pharmacy tasks based on the ranking of each of the ranked clinical opportunities.

4. The system of claim 1, wherein the first clinical opportunity is either initiated manually by one of a user selection on a user interface of the first remote pharmacy management system or initiated automatically when the first clinical opportunity becomes an active task in a workflow of the first remote pharmacy management system.

5. The system of claim 1, wherein integrating the ranked clinical opportunities into the workflow of the plurality of remote pharmacy management systems comprises injecting a respective subset of the ranked clinical opportunities between other pharmacy tasks in each respective workflow of the plurality of remote pharmacy management systems.

6. The system of claim 1, wherein the instructions further cause the system to filter the clinical opportunities by at least one of location, pharmacy identifier, priority setting, or store division before or directly after ranking.

7. The system of claim 1, wherein a user of a user device associated with the first remote pharmacy management system is automatically navigated to a user interface associated with the first clinical opportunity responsive to initiation of the first clinical opportunity.

8. The system of claim 1, wherein the plurality of sources comprise one or more clinical vendors.

9. The system of claim 1, wherein regularly or continuously updating the current status of the first clinical opportunity comprises setting the current status as one of pending, in progress, follow up, complete, deactivate, or available.

10. The method of claim 9, wherein the current status of each of the ranked clinical opportunities is set to pending after integration, and wherein the current status of the first clinical opportunity is set to in progress after initiation.

11. A method comprising:
aggregating clinical opportunities from a plurality of remote sources, wherein the clinical opportunities are patient care tasks to be performed by a pharmacy user;
ranking the clinical opportunities using one or more ranking parameters;
integrating the ranked clinical opportunities into a workflow of a plurality of remote pharmacy management systems;
determining that a first clinical opportunity from the ranked clinical opportunities has been initiated by a first remote pharmacy management system of the plurality of remote pharmacy management systems; and
updating a current status of the first clinical opportunity within the clinical opportunities repository once the first clinical opportunity is initiated.

12. The method of claim 10, wherein integrating the ranked clinical opportunities into the workflow of the plurality of remote pharmacy management systems comprises:
identifying a subset of the ranked clinical opportunities associated with each of the plurality of remote pharmacy management systems; and
distributing each subset of the ranked clinical opportunities to a respective one of the plurality of remote pharmacy management systems to be integrated with respective workflows of the one of the plurality of remote pharmacy management systems.

13. The system of claim 1, wherein integrating the ranked clinical opportunities into a workflow of one or more pharmacy management systems comprises injecting the ranked clinical opportunities between other pharmacy tasks based on the ranking of each of the ranked clinical opportunities.

14. The method of claim 10, wherein the first clinical opportunity is either initiated manually by one of a user selection on a user interface of the first remote pharmacy management system or initiated automatically when the first clinical opportunity becomes an active task in a workflow of the first remote pharmacy management system.

15. The method of claim 10, wherein integrating the ranked clinical opportunities into the workflow of the plurality of remote pharmacy management systems comprises injecting a respective subset of the ranked clinical opportunities between other pharmacy tasks in each respective workflow of the plurality of remote pharmacy management systems.

16. The method of claim 10, further comprising filtering the clinical opportunities by at least one of location, pharmacy identifier, priority setting, or store division before or directly after ranking.

17. The method of claim 10, wherein a user of a user device associated with the first remote pharmacy management system is automatically navigated to a user interface associated with the first clinical opportunity responsive to initiation of the first clinical opportunity.

18. The method of claim 10, wherein the plurality of remote sources comprise one or more clinical vendors.

19. The method of claim 10, wherein regularly or continuously updating the current status of the first clinical opportunity comprises setting the current status as one of pending, in progress, follow up, complete, deactivate, or available.

20. A non-transitory computer readable medium having instructions stored thereon that, when executed by one or more processors, cause a device to:
aggregate clinical opportunities from a plurality of remote sources, wherein the clinical opportunities are patient care tasks to be performed by a pharmacy user;
rank the clinical opportunities using one or more ranking parameters;
integrate the ranked clinical opportunities into a workflow of a plurality of remote pharmacy management systems;
determine that a first clinical opportunity from the ranked clinical opportunities has been initiated by a first remote pharmacy management system of the plurality of remote pharmacy management systems; and
update a current status of the first clinical opportunity within the clinical opportunities repository once the first clinical opportunity is initiated.
